(19) United States
(12) Patent Application Publication
(10) Pub. No.: US 2011/0027308 A1
(43) Pub. Date: Feb. 3, 2011

(54) COMPOSITIONS AND METHODS FOR PREVENTING INFLUENZA INFECTION IN CANINES, FELINES AND EQUINES

(75) Inventors: Teshome Mebatsion, Watkinsville, GA (US); Jessica Moore, Millsboro, DE (US); Perry R. Linz, Athens, GA (US)

Correspondence Address: Intervet/Schering-Plough Animal Health, Patent Dept. K-6-1, 1990, 2000 Galloping Hill Road, Kenilworth, NJ, 07033-0530, US

(73) Assignee: INTERVET INTERNATIONAL B.V.

(21) Appl. No.: 12/676,079

(22) PCT Filed: Sep. 9, 2008
(86) PCT No.: PCT/US08/75704

§ 371 Date: Mar. 2, 2010

Related U.S. Application Data
(60) Provisional application No. 60/971,096, filed on Sep. 10, 2007.

(51) Int. Cl. A61K 39/145; C12N 7/01; A61P 37/04; A61P 31/16

(52) U.S. Cl. 424/199.1; 435/235.1

(57) ABSTRACT

The present invention is directed to the use of recombinant, chimeric mononegavirale vectors comprising a foreign gene. More particularly, the present invention is directed to the use of such vectors for treating respiratory diseases in canines, felines or equines. An embodiment of the present invention is the use of a recombinant Newcastle disease virus vector comprising a hemagglutinin from an H3N8 influenza in the protection or treatment of canines, equines or felines against influenza.

### FIELD OF THE INVENTION

The present invention is directed to the use of recombinant, chimeric mononegavirale vectors comprising a foreign gene. More particularly, the present invention is directed to the use of such vectors for treating respiratory diseases in canines, felines, or equines.

### BACKGROUND OF THE INVENTION

Live viruses that are able to replicate in an infected host induce a strong and long-lasting immune response against their expressed antigens. They are effective in eliciting both humoral- and cell-mediated immune responses, as well as stimulating cytokine and chemokine pathways. Therefore, live, attenuated viruses offer distinct advantages over vaccine compositions based on either inactivated or subunit immunogens which typically largely only stimulate the humoral arm of the immune system.

Over the last decade recombinant DNA technology has revolutionized the field of genetic engineering of the genomes of both DNA and RNA viruses. In particular, it is now possible to introduce foreign genes into the genome of a virus such that upon replication of the new vector virus in a host animal a foreign protein is expressed that can exert biological effects in the host animal. As such, recombinant vector viruses have been exploited not only for the control and prevention of microbial infections, but also for devising target therapies for non-microbial diseases such as malignancies and in gene therapy.

The generation of non-segmented, negative stranded RNA viruses (viruses of the order Mononegavirales) entirely from cloned cDNA by a technique designated as “reverse genetics” was first described in U.S. Pat. No. 6,033,886, which is wholly incorporated by reference herein. This patent has made it possible to use viruses of the order Mononegavirales (MV) as vectors. Subsequent studies have been published that describe the use of viruses of the order MV as viral vectors to express foreign antigens derived from a pathogen aiming at developing vaccines against that pathogen.

The order of Mononegavirales is classified into four main families: Paramyxoviridae, Rhabdoviridae, Filoviridae and Bomaviridae. Viruses belonging to these families have genomes that are represented by a single, negative (−) sense RNA molecule, i.e. the polarity of the RNA genome is opposite to the polarity of messenger RNA (mRNA) that is designated as plus (+) sense. The classification of the main human and veterinary MV viruses is presented in the table below:

TABLE 1
Classification of main viruses within the order of Mononegavirales

| Family | Genus | Exemplary Species |
|---|---|---|
| Rhabdoviridae | Lyssavirus | Rabies virus (RV) |
| | Vesiculovirus | Vesicular stomatitis virus |
| | | (VSV) |
| | Novirhabdovirus | Infectious hematopoietic |
| | | necrosis virus (IHNV) |
| Paramyxoviridae | Respirovirus | Sendai virus (SeV) |
| | | Human parainfluenza virus |
| | | type 1 and type 3 (hPIV 1/3) |
| | | Bovine parainfluenza virus |
| | | type 3 (bPIV 3) |
| | Morbillivirus | Measles virus (MV) |
| | | Rinderpest virus |
| | | Canine distemper virus |
| | | (CDV) |
| | Rubulavirus | Simian virus 5 (SV-5) |
| | | Human parainfluenza virus |
| | | type 2 (hPIV 2) |
| | | Mumps virus |
| | Avulavirus | Newcastle disease virus |
| | | (NDV) |
| | Pneumovirus | Human respiratory syncytial |
| | | virus (hRSV) |
| | | Bovine respiratory syncytial |
| | | virus (bRSV) |
| Filoviridae | Ebola-like virus | Ebolavirus |
| | | Marburg virus |

The genomic organization and details of the life cycle of MV viruses is well known and has been reviewed by various authors. Although Mononegavirales viruses have different hosts and distinct morphological and biological properties, they have many features in common, such as genomic organization and the elements essential for their typical mode of replication and gene expression, illustrating that they have originated from a common ancestor. They are enveloped viruses that replicate in the cytoplasm of the cell and produce mRNAs that are not spliced.

A Mononegavirales virus consists of two major functional units, a ribonucleoprotein (RNP) complex and an envelope. The complete genome sequences for representative viruses of the genera of all the families mentioned above have been determined. The genomes range in size from about 9,000 nucleotides to about 19,000 and they contain from 5 to 10 genes. The structure and the organization of the genomes of the MV viruses are very similar and are governed by their particular mode of gene expression. All of the MV virus genomes comprise three core genes encoding: a nucleoprotein (N or NP), a phosphoprotein (P) and a RNA-dependent RNA polymerase (L). The viral envelope is composed of a matrix (M) protein and one or more transmembrane glycoproteins (e.g. G, HN and F proteins) that play a role in virus assembly/budding as well as in the cell attachment and/or entry of the virus. Depending on the genus, the protein repertoire is extended by accessory proteins that display certain specific regulatory functions in transcription and virus replication or that are involved in virus host reactions (e.g. C, V and NS proteins). The gene order of MV viruses is highly conserved with the core genes N and P, at or near the 3′ terminus and with the large (L) gene at the 5′ distal position. The M, the surface glycoprotein genes, as well as the other accessory genes, are located between the N, P and L genes.

In the RNP complex, the genomic or antigenomic RNA is tightly encapsilated with the N protein and is associated with the RNA-dependent RNA polymerase that consists of the L and P protein. After infection of a cell, the RNP complex, but not the naked RNA genome, serves as a template for two distinct RNA synthesis functions, i.e. transcription of subgenomic mRNAs and replication of full length genomic RNA.

All of the tandemly arranged genes are separated by so called “gene junction” structures. A gene junction comprises a conserved “gene end” (GE) sequence, a non-transcribed “intergenic region” (IGR) and a conserved “gene start” (GS) sequence. These sequences are both sufficient and necessary for gene transcription. During transcription each gene is sequentially transcribed into mRNA by the viral RNA-dependent RNA polymerase that starts the transcription process at the 3′ end of the genomic RNA at the first GS sequence. At each gene junction transcription is interrupted as a result of the disengagement of the RNA polymerase at the GE sequence. Re-initiation of transcription occurs at the subsequent GS sequence, although with a reduced efficiency. As a result of this interrupted process, also designated as a “stop-start” process, attenuation of transcription occurs at each gene junction as a result of which the 3′ proximal genes on a MV virus genome are transcribed more abundantly than successive down stream genes. The modular form of transcription of MV virus genes in which each gene is part of a separate cistron or transcription unit makes these viruses extremely well suited for the insertion and expression of foreign genes. Each transcription unit in a MV virus genome comprises the following elements: 3′-GS-open reading frame (ORF)-GE-5′.

At the 3′- and 5′-genomic termini all of the MV virus genomes have a short non-transcribed region called “leader” (about 40-50 nt) and “trailer” (about 20-600 nt), respectively. The leader and trailer sequences are essential sequences that control the replication of genomic RNA, viral encapsidation and packaging.

The reverse genetics technology and the rescue of infectious MV virus have made it possible to manipulate its RNA genome through its cDNA copy. Such techniques are referred to in U.S. Pat. No. 6,033,886, which is herein incorporated by reference in its entirety.

The minimal replication initiation complex required to synthesize viral RNA is the ribonucleoprotein (RNP) complex. Infectious MV virus can be rescued by intracellular co-expression of (anti)genomic RNAs and the appropriate support proteins from (T7) RNA polymerase driven plasmids. Reliable recovery of many MV virus species has been achieved based on the protocol described in U.S. Pat. No. 6,033,886 (or slight variations thereof).

Newcastle disease is an important disease of poultry, which can cause severe economic losses in the poultry industry worldwide. Newcastle disease virus is a non-segmented, negative stranded RNA virus within the order of MV. The genome, which is about 15 kb in length, contains six genes which encode the nucleoprotein (NP), phosphoprotein (P), matrix (M) protein, fusion (F) protein, hemagglutinin-neuraminidase (HN) protein and RNA-dependent RNA polymerase or large (L) protein. The NDV genes are arranged sequentially in the order 3′-NP-P-M-F-HN-L-5′, and are separated by intergenic regions of different length. All genes are preceded by a gene start (GS) sequence which is followed by a noncoding region, the open reading frame encoding the NDV proteins, a second noncoding region and the gene end (GE) sequence. The length of the NDV genome is a multiple of six which has to be considered for the introduction of foreign genes.

Influenza is a disease in dogs, horses and cats characterized by mild respiratory signs to severe disease with high mortality. Influenza has become endemic in dogs and horses. The canine influenza disease caused by an H3N8 influenza virus (CIV), is very closely related to equine H3N8 viruses, and all dogs are susceptible to infection. Approximately 80% of exposed dogs develop clinical signs. The causative agent is an H3N8 influenza A virus belonging to the family Orthomyxoviridae.

Canine and equine influenza is discussed further in U.S. Patent Application Nos. 60/673,443, filed Apr. 21, 2005; 11/409,416, filed Apr. 21, 2006; 60/779,080, filed Mar. 3, 2006; 60/728,449, filed Oct. 19, 2005; 60/754,881, filed Dec. 29, 2005; 60/759,162, filed Jan. 14, 2006; and 60/761,451, filed Jan. 23, 2006; and in international patent application no. PCT/US2006/015090, filed Apr. 21, 2006 (published as WO06/116082) all of which are herein incorporated by reference in their entirety. Feline influenza is discussed further in U.S. patent application No. 60/854,351 in the name of Lakshmanan et al., filed Oct. 25, 2006, which is herein incorporated by reference in its entirety.

Unlike Mononegavirales, influenza A virus contains eight genomic RNA segments of negative polarity which encode 10 proteins. Based on the antigenicity of the surface glycoproteins hemagglutinin (HA) and neuraminidase (N), influenza A viruses were subtyped. Up to now, 16 hemagglutinin (H 1-H 16) and nine neuraminidase (N 1-N 9) subtypes are known.

The reverse genetics technology and the rescue of such infectious segmented negative strand RNA viruses, such as influenza, have made it possible to manipulate such RNA genomes through cDNA copies. Such techniques are referred to in U.S. Pat. Nos. 6,544,785; 6,649,372; and 6,951,754, which are all herein incorporated by reference in their entirety. Such techniques are also referred to in various published U.S. patent applications having publication nos. 2004/0142003; 2003/035814; 2006/0057116; 2006/0134138; 2005/0186563, which are also all herein incorporated by reference in their entirety.

Different recombinant negative-strand RNA viruses expressing foreign proteins have been constructed. The S gene of severe acute respiratory syndrome and the F gene of respiratory syncytial virus have each been inserted into NDV for administration to primates. Hemagglutinins (HA) from different influenza subtypes have also been inserted into different vector viruses. For example, nucleic acids encoding HA from an H5N2 influenza has been inserted in infectious laryngotracheitis virus (ILTV) (Luschow et al., Vaccine 19, 4249-59, 2001); HA from an H3N2 influenza has been inserted in Rinderpest virus (Walsh et al., J. Virol. 74, 10165-75, 2000); and HA from an H1N1 has been inserted in vesicular stomatitis virus (VSV) (Roberts et al., J. Virol. 72, 4704-11, 1998).

NDV has also been used for the expression of hemagglutinin (or hemagglutinin derivatives) derived from H5N1, H5N2, and H7N7 influenza subtypes (Ge, J. et al. J. Virol 81(1): 150-8 (2007); Veits, J. et al., Proc. Natl. Acad. Sci. USA 103(21): 8197-202 (2006); Park, M. S. et al., Proc. Natl. Acad. Sci. USA 103(21): 8203-8 (2006)).

The hemagglutinin gene of influenza A/WSN/33 (H1N1) has also been inserted between P and M genes of NDV strain Hitchner B1. This recombinant protected mice against lethal infection although there was a detectable weight loss in the mice which recovered fully within 10 days (Nakaya et al., J. Virol. 75, 11868-73, 2001). A further recombinant NDV with the same insertion site for the foreign gene expressed the H7 of a low pathogenic AIV, but only 40% of the vaccinated chickens were protected from both velogenic NDV and highly pathogenic AIV (Swayne et al., Avian Dis. 47, 1047-50, 2003).

Thus far, use of NDV viruses encoding influenza genes has been limited in that the influenza genes are derived from H1-, H5- or H7-subtype influenza A hemagglutinin genes. Moreover, use of such recombinant negative strand viruses has been limited to and focused upon protection of poultry or primates against avian influenza. Prior to the present invention described herein, no work has been published describing the use of recombinant NDV having an inserted influenza A hemagglutinin gene to protect equines, canines or felines from influenza. Because dogs, horses and cats are susceptible to influenza, however, a need exists for such constructs that can serve as the basis for an influenza vaccine.

### SUMMARY OF THE INVENTION

The present invention relates to an immunogenic composition comprising a recombinant mononegavirale virus having a nucleotide sequence from an H3-type influenza or H3N8 influenza. The present invention also relates to an immunogenic composition comprising a recombinant mononegavirale virus having a nucleotide sequence from a canine influenza. The present invention also relates to an immunogenic composition comprising a recombinant mononegavirale virus having a nucleotide sequence from an equine influenza. The mononegavirale virus can be NDV and the influenza sequence can be a hemagglutinin sequence.

The present invention also relates to a method of protecting canines against respiratory infections comprising administering a recombinant mononegavirale virus having a heterologous respiratory viral nucleotide sequence. The present invention also relates to a method of protecting felines against respiratory infections comprising administering a recombinant mononegavirale virus having a heterologous respiratory viral nucleotide sequence. The present invention also relates to a method of protecting equines against respiratory infections comprising administering a recombinant mononegavirale virus having a heterologous respiratory viral nucleotide sequence. The sequence can be an influenza nucleotide sequence. The sequence can be a canine influenza sequence or an equine influenza sequence. The sequence can be an H3N8 influenza sequence. The influenza sequence can be a hemagglutinin influenza sequence. The recombinant mononegavirale virus can be intranasally administered. The recombinant mononegavirale virus can be a recombinant NDV virus.

### DETAILED DESCRIPTION OF THE INVENTION

The present invention relates to recombinant chimeric MV virus vectors containing a foreign, or heterologous, gene. The foreign gene can be a different strain of the same species of the MV virus vector. Alternatively, the foreign gene can be from a different viral species than the MV virus vector. More particularly, the present invention is directed to the use of such vectors for treating respiratory diseases in canines, felines or equines. An embodiment of the present invention is the use of a recombinant Newcastle disease virus vector comprising a hemagglutinin from an H3N8 influenza in the protection or treatment of canines, equines or felines against influenza.

Canines, equines and felines are susceptible to H3-, N8- and H3N8-subtype influenza A viruses. Additionally, canines, equines and/or felines can be susceptible to H5-, N1-, H5N1-, H3-, N8-, H3N8-, H7-, N7- and/or H7N7-subtype influenza A viruses. Because antibodies to the influenza H and N proteins are important in humoral immune response and inhibit infection or prevent disease, vaccines and use thereof according to the present invention can be based on one or more of the influenza A viral subtypes described above.

Methods for the preparation of such recombinant chimeric MV virus vector harboring an additional transcription unit comprising a foreign gene are well known in the art. For example, International Patent Application No. PCT/US07/64046, filed on Mar. 15, 2007 in the name of Römer-Oberdörfer et al., and U.S. Patent Application No. 60/783,194, filed on Mar. 15, 2006 in the name of Römer-Oberdörfer et al., (which are both wholly incorporated by reference herein) describe how to make such recombinant chimeric MV virus vectors comprising additional transcription unit having the foreign gene. These patent applications refer to documents that describe the preparation of such recombinant vector viruses for various MV virus species. In principle, the method used to make such recombinant chimeric MV virus vectors for use in the present invention is the same as that in the prior art. The foreign gene to be inserted into the MV virus genome can be flanked by the appropriate 3′- and 5′-non-coding regions, as defined by International Patent Application No. PCT/US07/64046 and U.S. Patent Application No. 60/783,194.

International Patent Application No. PCT/US07/64046 and U.S. Patent Application No. 60/783,194 describe how the presence of the 3′- and 5′ non-coding regions of a MV virus gene in a transcription unit comprising a foreign gene inserted into the genome of a MV virus have a positive effect on the transcription and/or expression of the foreign gene. It is shown in these patent applications that engineering the non-coding regions of a MV virus gene between a GS sequence and an avian influenza virus (AIV) hemagglutinin (HA) gene and between the AIV HA gene and a GE sequence increases the amount of HA mRNA synthesized by a MV virus vector harboring that AIV HA gene. These patent applications also show a positive effect on the protein expression level of the foreign (or, heterologous) gene. Therefore, the recombinant MV virus vector for use according to the present invention can be constructed such that non-coding regions as described in these patent applications flank the foreign gene.

Hence, recombinant, chimeric MV vectors for use in the methods of the present invention can be constructed such that the additional transcription unit comprising the foreign gene is operatively linked with an upstream MV virus gene start (GS) sequence and a downstream MV virus gene end (GE) sequence. Between the GS sequence and a start codon of the foreign gene can be located a 3′ non-coding region (genome sense) of a MV virus gene. Between a stop codon of the foreign gene and the GE sequence can be located a 5′ non-coding region (genome sense) of a MV virus gene.

The non-coding regions can be of a gene encoding a MV virus envelope protein, in particular a M, G, F or HN protein, or a RNP protein, in particular, a N, P or L protein. The GS and GE sequences to be used in this invention as the transcriptional control sequences are preferably those derived from the natural genes of the MV viruses. Without being limited to the following theory, it is believed that these sequences modulate the activity of the RNA polymerase during the transcription process, in particular in the process of transcription initiation and mRNA 5′ end modification and in control of transcription 3′ end polyadenylation and termination.

Detailed information of the genomic organization of MV viruses is known in the art, including the nucleotide sequences of the various MV virus genes and their transcription control (GS and GE) sequences and non-coding sequences that flank the genes. Such information is, for example, available from the U.S. Department of Health and Human Services' National Institutes of Health's National Library of Medicine's National Center for Biotechnology Information internet site. Some of these sequences are provided in International Patent Application No. PCT/U.S.07/64046, and in U.S. Patent Application No. 60/783,194, which are herein incorporated by reference in their entirety.

The recombinant, chimeric MV virus vector for use in the present invention can be prepared by inserting an isolated nucleic acid molecule comprising (i) a foreign gene flanked by the 3′- and 5′-non-coding regions as described above and (ii) the appropriate transcriptional control sequences, into the genome of the MV virus, such that in the resulting MV virus vector the foreign gene is both preceded and followed by a MV virus gene junction, in particular by a genomic nucleotide sequence fragment comprising GE-IGR-GS elements. The presence of such upstream and downstream elements guarantee the appropriate transcription not only of the inserted foreign genes, but also of the MV virus genes that are located up- and downstream of the inserted foreign gene.

The isolated nucleic acid molecule and the genome of the MV virus can be genetically manipulated in their cDNA form (+sense). This allows easy manipulation and insertion of the desired nucleic acid molecules into the viral genome. Various parts of the genome can be used for the insertion of the foreign gene, between two genes, i.e. in intergenic regions (IGR), 3′ or 5′ non-coding regions of a gene as well as 3′ promoter-proximal (before the N/NP genes) or 5′ distal end (after the L genes) of a genome.

The foreign gene could advantageously be inserted before the NP gene, between the NP and P genes, between the P and M genes, between the M and F genes, between the F and HN genes, between the HN and L genes, or after the L gene.

The preparation of such transcription cassettes and the insertion thereof into a MV virus genome only involve routine molecular biologic techniques, such as exemplified in International Patent Application No. PCT/U.S.07/64046, and in U.S. Patent Application No. 60/783,194. In particular, techniques such as site-directed- and PCR mutagenesis can be used for this purpose. More in particular, a recombinant MV virus vector according to the present invention can be prepared by means of the well established “reverse genetics” method that enables the genetic modification of non-segmented, negative stranded RNA viruses of the order MV, as described in U.S. Pat. No. 6,033,886, which is wholly incorporated by reference herein.

In this method, an appropriate cell is co-transfected by a vector comprising a cDNA molecule comprising a nucleotide sequence that encodes a full length genome, or, preferably, an antigenome (positive sense) of a MV virus, and one or more vectors comprising the cDNA molecules comprising nucleotide sequences that encode the required support proteins (i.e., to express functional viral RNA dependent RNA polymerase that can form the ribonucleoprotein complex), under conditions sufficient to permit transcription and co-expression of the MV (anti)genome and support proteins and the production of a recombinant MV vector. In this method, the nucleic acid molecule encoding the full length MV virus (anti)genome comprises an additional transcription unit as defined above.

Non-limiting examples of vectors for expressing viral polymerase or for making genomic RNAs include plasmids, phages or cosmids, to which another DNA segment may be attached so as to bring about the replication of the attached DNA segment and its transcription and/or expression in a cell transfected with this vector. For the intra-cellular expression of the appropriate support proteins use is made, preferably, of plasmids comprising the cDNA sequence encoding these proteins, under the control of appropriate expression control sequences, e.g., a T7 polymerase promoter.

Preferably, the vector for the transcription of the full length genome is a plasmid that comprises a cDNA sequence encoding the (anti)genome of the MV virus, flanked by a T7 polymerase promoter at its 5′ end and a (hepatitis delta) ribozyme sequence at its 3′ end, although a T3 or SP6 RNA polymerase promoter can also be used.

In a preferred embodiment of the invention, the recombinant MV virus vector for use according to the present invention is prepared using expression plasmids that encode the N (or NP), P and L proteins of the MV virus.

The amounts or ratios of transfected support plasmids to be used in this reverse genetic technology cover a broad range. Ratios for the support plasmids N:P:L may range from about 20:10:1 to 1:1:2 and efficient transfection protocols for each virus are known in the art.

An exact copy of the genomic RNA is made in a transfected cell by the combined action of the T7 RNA polymerase promoter and the ribozyme sequence, and this RNA is subsequently packaged and replicated by the viral support proteins supplied by the co-transfected expression plasmids.

The T7 polymerase enzyme can be provided by a recombinant vaccinia virus that infects the transfected cell, in particular by the vaccinia virus vTF7-3, yet also other recombinant pox vectors, such as fowl pox virus, e.g. fpEFLT7pol, or other viral vectors may be used for the expression of T7 RNA polymerase.

Separation of the rescued virus from vaccinia virus can easily be accomplished by simple physical techniques, such as filtration. Rescue of NDV can also be achieved by inoculation of the supernatant of transfected cells in embryonated eggs.

Alternatively, cell lines can be used for the transfection of the transcription- and expression vectors that constitutively express the (T7) RNA polymerase and/or one or more of the required support proteins.

Furthermore, more detailed information concerning the reverse genetics technology to be used herein for the preparation of a MV virus according to the present invention is disclosed in U.S. Pat. No. 6,033,886, which is wholly incorporated by reference herein.

In an embodiment of the present invention a recombinant MV virus vector is provided wherein the MV virus is a recombinant Newcastle disease virus (NDV) that expresses an antigen from a canine, equine or feline influenza. The canine, equine or feline influenza can be H3N8-subtype influenza A. General reverse genetics methods for the genetic manipulation of NDV have previously been disclosed by U.S. Pat. Nos. 6,146,642, 6,451,323, and 6,719,979, which are all herein incorporated by reference in their entirety.

A foreign gene can advantageously be introduced into a NDV genome at various positions as outlined in general for MV viruses above. In particular, in a recombinant NDV vector according to the invention, a foreign gene (as part of an appropriate transcription unit) can be inserted between the following NDV genes: between the NP and P genes, between the P and M genes, between the M and F genes, between the F and HN genes, between the HN and L genes and at the 3′ proximal- and 5′ distal locus, such as in the 3′ proximal, P-M, M-F and F-HN regions.

Furthermore, in a recombinant NDV vector according to the present invention the non-coding regions that flank the foreign gene can be derived from all naturally occurring NDV genes, in particular from the N, P, M, F or HN genes.

In an embodiment, the recombinant NDV vector mutant comprises a hemagglutinin (HA) gene of an influenza A virus, preferably of a canine, equine or feline influenza virus, more preferably of an H3, an N8 or an H3N8 influenza virus. Other examples of foreign genes useful in recombinant MV virus vectors according to the present invention include the hemagglutinin or the neuramidase gene or any other gene of the H3N8-, H7N7- or H5N1-subtype influenza type A species.

The MV vector virus can be attenuated, that is to say, the vector virus is not pathogenic for the target animal or exhibits a substantial reduction of virulence compared to the wild-type virus. For example, the NDV virus has only restricted replication in canines, equines and felines; and canines do not manifest clinical symptoms when exposed to the NDV virus. In addition, conventional techniques exist to obtain and screen for attenuated viruses that show a limited replication or infectivity potential. Such techniques include serial (cold) passaging the virus in a heterologous substrate and chemical mutagenesis. Such viruses are described in U.S. Pat. Nos. 6,177,082; 6,398,774; 6,482,414; 6,579,528; 7,029,903; and 7,074,414 which are all herein wholly incorporated by reference.

A recombinant NDV vector according to the invention can be derived from any conventional ND vaccine strain. Non-limiting examples of such suitable NDV strains are those present in commercially available NDV vaccines such as the Clone 30 strain (available in Combovac-30® vaccine, Intervet Inc., Millsboro, Del.), La Sota strains, Hitchner B1 strains, NDW stains, C2 strains and AV4 strains.

A recombinant MV virus vector according to the present invention is able to induce a protective immune response in animals. Therefore, in another embodiment of this invention a vaccine against a viral, microbial or parasitical pathogen is provided that comprises a recombinant MV virus vector as defined above in a live or inactivated form, and a pharmaceutically acceptable carrier or diluent.

A vaccine according to the invention can be prepared by conventional methods such as those commonly used for the commercially available live- and inactivated MV virus vaccines. A susceptible substrate can be inoculated with the recombinant MV virus vector and propagated until the virus replicated to a desired titre after which the virus containing material is harvested. Subsequently, the harvested material is formulated into a pharmaceutical preparation with immunizing properties.

Every substrate which is able to support the replication of the recombinant MV virus vector can be used in the present invention. As a substrate host cells can be used from both prokaryotic- and eukaryotic origin, depending on the MV virus. Appropriate host cells may be vertebrate, e.g. primate cells. Suitable examples are; the human cell lines HEK, WI-38, MRC-5 or H-239, the simian cell line Vero, the rodent cell line CHO, BHK, the canine cell line MDCK or avian CEF or CEK cells.

A particularly suitable substrate on which a recombinant NDV vector according to the present invention can be propagated are SPF embryonated eggs. Embryonated eggs can be inoculated with, for example 0.2 ml NDV containing allantoic fluid comprising at least 10^{2.0} EID₅₀ per egg. Preferably, 9- to 11-day old embryonated eggs are inoculated with about 10^{5.0} EID₅₀ and subsequently incubated at 37° C. for 2-4 days. After 2-4 days the ND virus product can be harvested preferably by collecting the allantoic fluid. The fluid can be centrifuged thereafter for 10 minutes at 2500g followed by filtering the supernatant through a filter (100 μm).

The vaccine according to the invention comprises the recombinant MV virus vector together with a pharmaceutically acceptable carrier or diluent customarily used for such compositions. The vaccine containing the live virus can be prepared and marketed in the form of a suspension or in a lyophilized form. Carriers include stabilizers, preservatives and buffers. Diluents include water, aqueous buffer and polyols.

In another aspect of the present invention a vaccine is provided comprising the recombinant MV virus vector in an inactivated form. The major advantages of an inactivated vaccine are its safety and the high levels of protective antibodies of long duration that can be induced. The aim of inactivation of the viruses harvested after the propagation step is to eliminate reproduction of the viruses. In general, this can be achieved by well known chemical or physical means.

If desired, the vaccine according to the invention may contain an adjuvant. Examples of suitable compounds and compositions with adjuvant activity for this purpose are aluminum hydroxide, -phosphate or -oxide, oil-in-water or water-in-oil emulsion based on, for example a mineral oil or a vegetable oil such as vitamin E acetate, and saponins.

Administration of the vaccine to a subject results in stimulating an immune response in the subject mammal. The route of administration for vaccines to the mammalian target can be achieved according to methods known in the art. Such methods include, but are not limited to, intradermal, intramuscular, intraocular, intraperitoneal, intravenous, parenteral, intranasal, oral, oronasal, and subcutaneous, as well as inhalation, suppository, or transdermal. The vaccine may be administered by any means that includes, but is not limited to, syringes, nebulizers, misters, sprays, needleless injection devices, or microprojectile bombardment gene guns (balistic bombardment).

Recombinant NDV vector vaccine for use according to the invention is preferably administered by the same inexpensive mass application techniques commonly used for NDV vaccination. For NDV vaccination these techniques include drinking water and spray vaccination.

The scheme of the application of the vaccine according to the invention to the target mammalian may be in single or multiple doses, which may be given at the same time or sequentially, in a manner compatible with the dosage and formulation, and in such an amount as will be immunologically effective.

A vaccine according to the invention comprises an effective dosage of the recombinant MV virus vector as the active component, i.e. an amount of immunizing MV virus material that will induce immunity in the vaccinated canines, felines or equines against challenge by a virulent virus or microbial or parasitical organism. Immunity is defined herein as the induction of a significant higher level of protection in a population of animals against mortality and clinical symptoms after vaccination compared to an unvaccinated group. In particular, the vaccine according to the invention prevents a large proportion of vaccinated animals against the occurrence of clinical symptoms of the disease and mortality.

Typically, the live vaccine can be administered in a dose of 10^{2.0}-10^{8.0} tissue culture/embryo infectious dose (TC/EID₅₀), preferably in a dose ranging from 10^{4.0}-10^{7.0} TC/EID₅₀. Inactivated vaccines may contain the antigenic equivalent of 10^{4.0}-10^{9.0} TC/EID₅₀.

The invention also includes combination vaccines comprising, in addition to the recombinant MV virus vector according to the invention, a vaccine capable of inducing protection against a further pathogen. Hence, the recombinant MV virus vector according to the present invention may be formulated in a vaccine comprising one or more additional immunogens. The additional immunoactive component(s) may be whole parasite, bacteria or virus (inactivated or modified live), or a fractionated portion or extract thereof (e.g., proteins, lipids, lipopolysacharide, carbohydrate or nucleic acid).

Where the recombinant MV virus vector according to the present invention is used in a canine vaccine, antigens for other canine pathogens may be added into the formulation. Non-limiting examples of other pathogens for which additional antigens may be added include Bordetella bronchiseptica, canine distemper caused by canine distemper virus (CDV), infectious canine hepatitis (ICH) caused by canine adenovirus type 1 (CAV-1) virus, respiratory disease caused by canine adenovirus type 2 (CAV-2) virus, canine parainfluenza caused by canine parainfluenza (CPI) virus, enteritis caused by canine coronavirus (CCV) and canine parvovirus (CPV), measles virus, and leptospirosis caused by Leptospira bratislava, Leptospira interrogans serovar canicola, Leptospira grippotyphosa, Leptospira icterohaemorrhagiae or Leptospira pomona, rabies virus, Hepatozoon canis and Borrelia burgdorferi, Canine Rota Virus (CRV), Canine Herpesvirus (CHV), Minute Virus of Canines (MVC), Giardia species, Babesia gibsoni, B. vogeli, B. rossi, Giardia and Salmonella species, and Leishmania donovani-complex.

Where the recombinant MV virus vector according to the present invention is used in an equine vaccine, antigens for other equine pathogens may be added into the formulation. Non-limiting examples of other pathogens for which additional antigens may be added include Eastern encephalomyelitis virus, Western encephalomyelitis virus, Venezuelan encephalomyelitis virus, equine herpes virus type 1, equine herpes virus type 4, equine influenza virus (e.g., strain KY93/A2, KY02/A2, and NM/2/93/A2), west nile virus, rhinopneumonitis virus, rabies virus, Streptococcus equi, Clostridial species (e.g., C. botulinum), Ehrlichia risticii, E. coli, Rhodococcus equi, rotavirus, Salmonella species, Sarcocystis neurona, and a tetanus toxoid fractions.

Where the recombinant MV virus vector according to the present invention is used in a feline vaccine, antigens for other feline pathogens may be added into the formulation. Non-limiting examples of other pathogens for which additional antigens may be added include rhinotracheitis virus, calicivirus, panleukopenia virus, Chlamydia (including C. psittaci), bordetella bronchiseptica, Giardia species, feline immunodeficiency virus, feline leukemia virus or rabies virus.

Alternatively, a vaccine based upon recombinant MV according to the present invention may be administered simultaneously or concomitantly with other live or inactivated vaccines.

The following examples are provided for illustrative purposes only, and are in no way intended to limit the scope of the present invention. The examples described below show the efficacy of a recombinant NDV expressing the HA gene of an H3N8 influenza virus after vaccination of dogs by intranasal or SC routes.

### EXAMPLES

### Example 1

### Construction of Recombinant NDV Expressing Canine Influenza Hemagglutinin Gene

A. Preparation of DNA Encoding Influenza Hemagglutinin (HA)

The influenza HA insert was obtained by reverse transcriptase polymerase chain reaction (RT-PCR) using the Access RT-PCR System kit (Promega Corporation, Madison, Wis.) The HA insert was amplified using viral isolate A/Canine/Jacksonville/05 (also known as canine/Jax/05, deposit no. PTA-7941, American Type Culture Collection (ATCC), 10801 University Blvd, Manassas, Va. 20110-2209) using hemagglutinin-specific primers (5′-acggcaatcgATGAAGACAACCATTATTTTGA-3′) and (5′-acggcagcgcgcTCAAATGCAAATGTTGCATC-3′) on a Robocycler® microprocessor controlled robotic temperature cycler (Stratagene, Santa Clara, Calif.) with Tfl DNA polymerase from the kit.

The amplified HA sequence was separated from other components of the PCR mixture on an electrophoretic gel and isolated. The purified HA sequence was cloned into pCRII-TOPO® (Invitrogen, Carlsbad, Calif.) according to the vector kit's manufacturer's instructions. The resulting plasmid, pCRII:CB1, contained the amplified influenza HA sequence. The hemagglutinin sequence was verified with Big-Dye™ terminator cycle sequencing (Applied Biosystems, Foster City, Calif.) and analysed using an Applied Biosystems 3100-Avant Capillary Sequencer.

B. Cloning Influenza HA Gene Sequence into Intermediate Plasmid

The HA gene sequence was first excised from pCRII:CB1 and sub-cloned into the intermediate plasmid pM3E. The generation of the recombinant pM3E intermediate plasmid has been described previously (Engel-Herbert, I. et al., “Characterization of a recombinant Newcastle disease virus expressing the green fluorescent protein,” J. Virol. Methods. 108:19-28 (2003)).

In brief, pM3E is derived from a full-length cDNA clone based on the vaccine strain Clone-30 (described in greater detail below). A Dra I-Nhe I fragment containing NDV sequences from nt 5233 to nt 6559 was cloned into the SmaI site of pUC18 (GenScript Corporation, Piscataway, N.J.) after treating with DNA polymerase I (Klenow). Then, the intergenic region (igr) between F and HN genes (situated from nt 6290 to nt 6320) was modified to contiain a PacI-restiction site at nt position 6300 (6296-6303) by site-directed mutagenesis using the primer 5′-gagagttaagaaaaaactaccgttaattaatgaccaaaggacg-3′. The resulting plasmid was named pM1E. Exchange of the F-HN igr of Clone-30 by the new igr containing the Pac I-site was performed using PshA I-sites. The introducte the transcription start and stop signals, two complementary oligonucleotides (5′ cgattaattaaacgggtagaagcgatcgcacgcgttaagaaaaattaattaacag and 5′-ctgttaattaattttttcttaacgcgtgcgatcgcttctacccgtttaattaatcg (having Pac I- and Mlu I-steds) containing synthetic gene-start (GS) and gene-end (GE) signals were introduced after Pac I digestion of pM1E to give rise to plasmid pM3E.

pM3E was prepared for hemagglutinin insertion by Mlu I digestion, blunt-ending with Klenow enzyme, and gel-purification, followed by dephosphorylation with calf intestinal alkaline phosphatase. To prepare for insertion into pM3E, the hemagglutinin gene was excised by digestion with Spe I and partial digestion with Cla I, blunt-ended with Klenow enzyme, and the resulting ˜1.7 kb fragment was excised and purified. Prepared hemagglutinin encoding DNA was cloned into prepared pM3E intermediate vector to generate the intermediate recombinant plasmid M3E:CI.

C. Cloning Influenza HA Gene Sequence into the Clone 30 cDNA

NDV Clone 30 strain is commercially available in Combovac-30® vaccine (Intervet Inc., Millsboro, Del.). The skilled artisan is familiar with the process of isolating RNA from this commercially available virus and generating a full length cDNA clone of the NDV Clone 30 strain. The construction of such a full-length cDNA clone has been described previously. See Römer-Oberdörfer, A., et al., “Generation of recombinant lentogenic Newcastle disease virus from cloned cDNA,” J. Gen. Vir. 80:2987-2995 (1999).

In brief, cDNA synthesis and assembly of a full-length clone of NDV strain Clone-30 is as follows. NDV strain Clone 30 is a vaccine strain derived from the lentogenic NDV strain of La Sota, and is available from Intervet Inc., Millsboro, Del. NDV strain Clone-30 was purified from 50 ml of allantoic fluid with a titre of 10¹⁰ egg-infectious doses (EID₅₀)/ml. After clarification of the allantoic fluid, NDV was sedimented by ultracentrifugation (1.5 h, 4° C., 150000 g), and viral RNA was isolated by guanidinium isothiocyanate extraction and subsequent centrifugation through a CsCl cushion. cDNA to genomic RNA was generated by two specifically primed cDNA syntheses (first strand reaction, Time Save cDNA synthesis kit, Pharmacia) with primers P1F (Clone-30 nt 1-21 of European Molecular Biology Laboratory-European Bioinformatics Institute (EMBL-EBI) accession no. Y18898) and P5F (Clone-30 nt 8326-8346). A third first strand reaction was carried out with primer P7F (Clone-30 nt 12736-12754) using SuperScript II RNase H-Reverse Transcriptase (Gibco-BRL). Subsequently, the reaction mixes were incubated with 2 units RNase H (Biozym) for 20 min at 3TC. After heat inactivation (5 min, 70° C.), the first strand cDNA was purified by phenol-chloroform extraction and ethanol precipitation. Each first strand cDNA was redissolved in a total of 20 μl H₂O, Subsequently, PCR was carried out on 1 μl of the first strand cDNA using the Expand High Fidelity (HF) PCR system (Boehringer Mannheim) with primer pair P2FMluI (5′ TGTGAATCACGCGTGCGAGCCCGA 3′, Clone-30 nt 68-91, MluIsite underlined, nts differing from the Clone-30 consensus sequence in bold) and P3R (5′ GACAAGCGGAAGAGCCATGCAAACTTGGCTGTG 3′, Clone-30 nt 8911-8890, synthetic adapter containing a SapI recognition site, underlined, and additional nucleotides in italics) to obtain fragment N1 (Clone-30 nt 1-8911) and primer pair P5F (Clone-30 nt 8326-8346) and P6R (Clone-30 nt 12919-12900) for the generation of fragment N2 (Clone-30 nt 8326-12919). Fragment N3 (Clone-30 nt 12736-15058) was generated by HF-PCR using primer pair P7F (Clone-30 nt 12736-12754) and P8RMluI (5′GTATAATTAAATCAACGCGTATACAA 3′, Beaudette C nt 15058-15033; MluI site underlined, nts differing from the Clone-30 consensus sequence in bold) from a cloned NDV fragment (nt 12736-15073) which had been obtained previously by RT-PCR. The terminal sequences of the genomic RNA were determined by polyadenylation of RNA and amplification of the 3′ end, and amplification of the 5′ end using the 5″-RACE protocol (Gibco BRL) as described in Mundt, E. et al., Virology 209: 10-18 (1995). Specific oligonucleotides were deduced to amplify PCR fragments which include leader and trailer, respectively, with primers P4FT7 (5 ′CTGAAGCTTGTAATACGACTCACTATAGGGACCAAACAGAGAATCCGT AAG 3′, HindIII site underlined, T7 promoter and three additional G residues in bold, Clone-30 nt 1-21) and P2RMluI (5′ TCGGGCTCGCACGCGTGATTCACACCT 3′, Clone-30 nt 91-65), and P8FMluI (5′ TTGTATACGCGTTGATITTATCATATTATG 3′, Clone-30 nt 15033-15062) and P9R TACTGCAGGGAAGACGTACCAAACAAAGATTTGGTGAA 3′, synthetic adapter, in italics, containing PstI/BbsI recognition sites underlined, Clone-30 nt 15186-15166) by HF-PCR, introducing two artificial MluI sites (underlined, nt 76,1 NP gene noncoding region, and nt 15039, L gene noncoding region) by mutation of five nucleotides (bold) (clone-30 nt 76 T to A, nt 79 A to C, nt 15039 T to A, nt 15041 T to G and nt 15042 T to C). In a multi-step cloning procedure, a complete NDV antigenome expression plasmid (pflNDV-1) was assembled in the SmaI site of pX8δT, as described in Schnell et al., EMBO J. 13:4195-4203 (1994), from cloned PCR fragments described above using naturally occurring restriction sites and the artificial MluI sites.

The hemagglutinin gene flanked by NDV transcription start and stop signals (M3E:CI) was excised by Pad digestion and ligated into the full-length clone of NDV strain Clone-30 containing the unique Pad site resulting in the full-length clone NDV:CI.

D. Transfection of NDV:CI into Mammalian Cells and Recovery of Recombinant NDV

Transfection experiments were carried out as described previously in Römer-Oberdörfer, A., et al., “Generation of recombinant lentogenic Newcastle disease virus from cloned cDNA,” J. Gen. Vir. 80:2987-2995 (1999). In brief, transfection experiments were done using BHK 21 cells, clone BSR T7/5, stably expressing the phage T7 RNA polymerase, as described in Buchholz, U. J. et al., J. Virol. 73(1):251-9 (1999). Cells were grown to ˜70% confluency in 32 mm diameter dishes and were transfected with a total amount of 20 μg DNA (5 μg of NP DNA, 2.5 μg of P DNA, 2.5 μg of L DNA and 10 μg of full length DNA) (Minis Trans IT transfection kit, Minis Corporation, Madison, Wis.).

48 to 72 hours after transfection into the cells, supernatants were harvested. After clearing cell debris by low-speed centrifugation, a volume of 700 μl of the supernatant was inoculated into the allantoic cavity of 10-day-old embryonated SPF chicken eggs. About 200 μl inoculum volume of allantoic fluid was used in subsequent egg passages. Hemagglutination tests were performed using 5% chicken erythrocytes with allantoic fluid harvested after the second 72 hr egg passage. HA positive allantoic fluid was used for further egg passages.

E. Confirmation of Expression of H3N8 Influenza Hemagglutinin in Recombinant NDV

Immunofluoresence Assay (IFA) Experiments: Baby hamster kidney cells (BSR cells) were cultured in media supplemented with 5% fetal bovine serum. Cells were infected with 0.5-2 μA allantoic fluid collected from each egg passage for 16-24 hours, followed by fixation with 100% ethanol. The presence of NDV antigen was assayed by using the monoclonal antibody NDV-57, directed toward the F protein and FITC-conjugated goat anti-mouse IgG (Kirkegaard and Perry Laboratories Inc., Gaithersburg, Md.). The expression of hemagglutinin of Canine H3N8 influenza virus was assayed using convalescent sera obtained from dogs challenged with A/Canine/Florida/242/2003 canine influenza viral isolate (deposit no. PTA-7915, American Type Culture Collection (ATCC), 10801 University Blvd, Manassas, Va. 20110-2209) and FITC-conjugated goat anti-dog IgG (Kirkegaard and Perry Laboratories Inc., Gaithersburg, Md.).

Western Blot analysis: Recombinant and parental Newcastle disease viruses were collected by ultracentrifugation of infected allantoic fluid. Canine influenza virus (A/Canine/Florida/242/2003) was obtained from the stock used to generate the polyclonal antisera. Recombinant and parental Newcastle virus was detected in a Western blot using the anti-NP monoclonal NDV-36 and HRP-conjugated goat anti-mouse IgG (Kirkegaard and Perry Laboratories Inc., Gaithersburg, Md.), and the Canine influenza antigens were detected in a Western blot using the anti-virus dog polyclonal and HRP-conjugated goat anti-dog IgG (Kirkegaard and Perry Laboratories Inc., Gaithersburg, Md.).

### Example 2

### Canine Influenza Vaccine Efficacy Study

In the following study, a method of protecting canines against respiratory infections comprising administering a recombinant mononegavirale virus having a heterologous respiratory viral nucleotide sequence was investigated.

Recombinant virus was passed in eggs a total of seven times and the allantoic fluid collected. EID₅₀ titers were determined by inoculating 200 μl 10-fold serial dilutions of P7 allantoic fluid into 10 embryonated 10 day of age eggs/dilution. Allantoic fluid was tested for agglutination after 5 days incubation. EID₅₀ titrations were calculated. Vaccine was prepared by diluting the allantoic fluid 10-fold in sterile phosphate buffered saline to an approximate titer of 10⁸ virions/ml, aliquotted, and frozen.

Each dose of vaccine contained 10⁸ EID₅₀ and sufficient PBS to bring the total dose volume to 1 ml.

Fifteen 1 yr+old female beagles were used in this study. Beagles were randomly assigned to three groups (Table 2). All dogs were fed with a standard growth diet and water was available ad libitum.

TABLE 2
Experimental design

| | | Vaccination | Number | | |
|---|---|---|---|---|---|
| Group | Treatment | route | of dogs | Dose | Challenge |
| 1 | NDV:CI | SC | 5 | 10⁸ EID₅₀ | Yes |
| | | | | in 1.0 ml | |
| 2 | NDV:CI | IN | 5 | 10⁸ EID₅₀ in | Yes |
| | | | | 1.0 ml (0.5 ml/ | |
| | | | | nostril) | |
| 3 | Control | N/A | 5 | NA | Yes |

Dogs in Group 1 received two doses of a recombinant Newcastle Virus expressing Canine Influenza Hemagglutinin at a 4 week interval by the subcutaneous route. Dogs in Group 2 received two doses of a recombinant Newcastle Virus expressing Canine Influenza Hemagglutinin at a 4 week interval by the intranasal route. Dogs in Group 3 were used as challenge control animals to assess clinical signs following Influenza virus challenge. Seven days before challenge, the dogs were moved to a BSL-2 facility and housed in individual cages All vaccinates and control dogs were challenged oronasally with virulent canine influenza virus (10⁷² TCID₅₀ of A/Canine/Florida/242/2003 per dog) at 2 weeks post second vaccination. The challenge virus was administered as a mist (2 ml/dog) using a nebulizer. The dogs were observed for influenza-related clinical signs for 14 days after challenge administration. Nasal and orapharyngeal swabs were collected daily in tubes containing 2 ml of virus transport medium for virus isolation from day −1 (i.e., one day before challenge) through day 14 post-challenge. The clinical signs observed during post challenge observations are shown in Table 3.

Serum samples were collected from all dogs on the day of first vaccination, on the day of challenge, and 14 days post-challenge to determine the HI titers using an H3N8 equine influenza virus standard protocol (Supplemental Assay Method (SAM) #124, Center for Veterinary Biologics (CVB), U.S. Dept. of Agriculture (USDA), Ames, Iowa).

Results: All dogs in Group 1 (subcutaneous, SC) developed HI antibody titers in response to the subcutaneous administration of the vaccine construct. Dogs in Groups 2 and 3 did not develop any titer against HI titers prior to challenge (Table 4). All dogs developed increased HI antibody titers in response to the challenge, with the highest average titer belonging to Group 1. Dogs in all groups exhibited one or more of the following clinical symptoms of canine influenza: fever (≧103.0° F.; ≧39.4° C.), cough, sneezing, ocular and nasal discharge, vomiting, and anorexia. However, the number of dogs and the number of symptom occurrences considerably differed between groups (Table 5). Dogs that received the recombinant vaccine intranasally (Group 2) had remarkably milder and a shorter duration of influenza-associated clinical signs when compared to Group 1 vaccinates and Group 3 controls (Table 5).

Virus isolation results are shown in Table 6. Following a virulent canine influenza virus challenge, the canine influenza virus was isolated from nasal swabs from 5 of 5 (100%) dogs from Group 1 (NDV:CI SC), 3 of 5 (60%) dogs from Group 2 (NDV-CI IN), and 5 of 5 (100%) controls (Group 3). Canine influenza virus was isolated oropharyngeal swabs from 2/5 (40%) of the dogs in Groups 1 and 3 (NDV:CI SC and Controls, respectively) and 0/5 (0%) dogs in Group 2. Intranasal administration of the recombinant vaccine demonstrated an overall 40% reduction in viral shedding in both the oral and nasal secretory pathways when compared to the non-vaccinated control group (Table 6).

Conclusion: The results from this study demonstrate that: (1) a recombinant mononegavirale virus (NDV in this case) having a heterologous respiratory viral nucleotide sequence (H3 in this instance) can be administered as an efficacious intranasal vaccine to canines, and presumably felines and equines, against respiratory pathogens, (2) Intranasal use of a recombinant NDV:H3 equine influenza virus or canine influenza virus vaccine can reduce the severity of canine influenza virus disease in dogs, and (3) Intranasal application of an NDVH3 equine or canine influenza virus vaccine can substantially reduce virus excretion in nasal and/or oral secretions.

TABLE 3
Clinical signs

Clinical signs
Rectal Temperature
Coughing
Phlegm
Nasal Discharge
Ocular Discharge
Sneezing
Gastro-intestinal Abnormalities
Anorexia
Vomiting
Fecal abnormalities

TABLE 4
Serology - Hemagglutination inhibition titers

| | Dog ID | Pre 1st Vac | Pre challenge | Necropsy |
|---|---|---|---|---|
| Vaccine Group | number | Day 1 | Day 42 | Day 56 |
| 1 | 288 | <10 | 80 | ≧640 |
| vaccinated | 583 | <10 | 20 | ≧640 |
| NDVpacCI.2 | 604 | <10 | 20 | ≧640 |
| 101706 | AID | <10 | 20 | ≧640 |
| SC route | AGI | <10 | 10 | ≧640 |
| 2 | 585 | <10 | <10 | 320 |
| vaccinated | 586 | <10 | <10 | 320 |
| NDVpacCI.2 | 591 | <10 | <10 | ≧640 |
| 101706 | 592 | <10 | <10 | ≧640 |
| IN route | AHD | <10 | <10 | ≧640 |
| 3 | 287 | <10 | <10 | 80 |
| Neg Con | 584 | <10 | <10 | 160 |
| for Chall | AGH | <10 | <10 | ≧640 |
| | AIJ | <10 | <10 | 320 |
| | AKG | <10 | <10 | ≧640 |
| +con sera | lot: | 320 | 320 | 320 |
| | 111406 | | | |
| −con sera | lot: | <10 | <10 | <10 |
| | 111406 | | | |

* First vaccination
** Second vaccination
*** Day of challenge

TABLE 5
Clinical Observations: Canine Influenza Post-Challenge

| | | | | | | | | Gastro- |
|---|---|---|---|---|---|---|---|---|
| | | Temp | | | Nasal | Ocular | | intestinal |
| Group | Treatment | >103.0° F. | cough | phlegm | Discharge | Discharge | sneezing | Abnormalities |
| 1 | NDV:CI | 2/5 | 4/5 | 1/5 | 3/5 | 1/5 | 3/5 | 2/5 |
| | SC | (40) | (80) | (20) | (60) | (20) | (60) | (40) |
| 2 | NDV:CI | 1/5 | 2/5 | 0/5 | 0/5 | 0/5 | 0/5 | 1/5 |
| | IN | (20) | (40) | (0) | (0) | (0) | (0) | (20) |
| 3 | control | 2/5 | 4/5 | 3/5 | 0/5 | 0/5 | 1/5 | 1/5 |
| | | (40) | (80) | (60) | (0) | (0) | (20) | (20) |

*Numbers in parenthesis indicate percentage of animals showing clinical signs.

TABLE 6
Post-Challenge Virus Shedding Results

| | | | Orapharyngeal |
|---|---|---|---|
| Group | Treatment | Nasal Swab | Swab |
| 1 | NDV:CI SC | 5/5 | 2/5 (40%) |
| 2 | NDV:CI IN | 3/5 | 0/5 (0%) |
| 3 | Control | 5/5 | 2/5 (40%) |

1. An immunogenic composition comprising a recombinant mononegavirale virus having a nucleotide sequence from an H3N8 influenza.

2-16. (canceled)

17. An immunogenic composition comprising a recombinant mononegavirale virus that comprises a heterologous nucleotide sequence from a H3N8 influenza virus encoding an immunogenic antigen.

18. The composition of claim 17, wherein the H3N8 virus is a canine virus.

19. The composition of claim 17, wherein the H3N8 virus is an equine virus.

20. The composition of claim 17, wherein the mononegavirale virus is NDV.

21. The composition of claim 17, wherein the H3N8 influenza sequence is a hemagglutinin sequence.

22. A method for protecting a canine against respiratory infection, comprising administering an immunogenic composition according to claim 17.

23. A method for protecting a feline against respiratory infection, comprising administering an immunogenic composition according to claim 17.

24. A method for protecting an equine against respiratory infection, comprising administering an immunogenic composition according to claim 17.

25. The method of claim 22, wherein the H3N8 influenza sequence is a canine influenza sequence.

26. The method of claim 23, wherein the H3N8 influenza sequence is a feline influenza sequence.

27. The method of claim 24, wherein the H3N8 influenza sequence is an equine influenza sequence.

28. The composition of claim 17, wherein the H3N8 influenza sequence is an influenza hemagglutinin sequence and the mononegavirale virus is NDV.

29. The method of claim 22, wherein the immunogenic composition is administered intranasally.

30. The method of claim 23, wherein the immunogenic composition is administered intranasally.

31. The method of claim 24, wherein the immunogenic composition is administered intranasally.

32. The method of claim 22, wherein the mononegavirale virus is a recombinant NDV virus.

33. The method of claim 23, wherein the mononegavirale virus is a recombinant NDV virus.

34. The method of claim 24, wherein the mononegavirale virus is a recombinant NDV virus.


SEQUENCE LISTING (12 sequences)

SEQ ID NO: 1 - DNA, 32 bp, artificial
  primer #1 from Example 1A
acggcaatcgatgaagacaaccattattttga

SEQ ID NO: 2 - DNA, 32 bp, artificial
  primer #2 from Example 1A
acggcagcgcgctcaaatgcaaatgttgcatc

SEQ ID NO: 3 - DNA, 43 bp, artificial
  primer #1 from Example 1B
gagagttaagaaaaaactaccgttaattaatgaccaaaggacg

SEQ ID NO: 4 - DNA, 56 bp, artificial
  primer #2 from Example 1B
cgattaattaaacgggtagaagcgatcgcacgcgttaagaaaaaattaattaacag

SEQ ID NO: 5 - DNA, 56 bp, artificial
  primer #3 from Example 1B
ctgttaattaattttttcttaacgcgtgcgatcgcttctacccgtttaattaatcg

SEQ ID NO: 6 - DNA, 24 bp, artificial
  primer #1 from Example 1C
tgtgaatcacgcgtgcgagcccga

SEQ ID NO: 7 - DNA, 33 bp, artificial
  primer #2 from Example 1C
gacaagcggaagagccatgcaaacttggctgtg

SEQ ID NO: 8 - DNA, 26 bp, artificial
  primer #3 from Example 1C
gtataattaaatcaacgcgtatacaa

SEQ ID NO: 9 - DNA, 51 bp, artificial
  primer #4 from Example 1C
ctgaagcttgtaatacgactcactatagggaccaaacagagaatccgtaag

SEQ ID NO: 10 - DNA, 27 bp, artificial
  primer #5 from Example 1C
tcgggctcgcacgcgtgattcacacct

SEQ ID NO: 11 - DNA, 30 bp, artificial
  primer #6 from Example 1C
ttgtatacgcgttgattttatcatattatg

SEQ ID NO: 12 - DNA, 38 bp, artificial
  primer #7 from Example 1C
tactgcagggaagacgtaccaaacaaagatttggtgaa